(19) United States
(12) Patent Application Publication
(10) Pub. No.: US 2020/0160877 A1
(43) Pub. Date: May 21, 2020

(54) METHOD AND SYSTEM FOR PROCESSING AUDIO SIGNALS FOR A MICROPHONE OF AN AIRCRAFT OXYGEN MASK

(71) Applicant: Airbus Operations SAS, Toulouse (FR)

(72) Inventors: Benoît GAUDUIN, TOULOUSE (FR); Nicolas CLEMENT, TOULOUSE (FR); Benoît DIONNET, TOULOUSE (FR)

(21) Appl. No.: 16/687,053

(22) Filed: Nov. 18, 2019

(30) Foreign Application Priority Data
Nov. 20, 2018 (FR) 1871614

(51) Int. Cl. G10L 21/0232; G06N 3/04; A61B 7/00; A62B 18/02; B64D 11/00

(52) U.S. Cl. CPC G10L 21/0232; G06N 3/0445; G10L 2021/02163; A62B 18/02; B64D 11/0015; A61B 7/003

(57) ABSTRACT

An audio signal processing system for a microphone of an aircraft oxygen mask receives audio signals captured by the oxygen mask microphone. The audio signal processing system comprises: a module for detecting breathing noise within the audio signals comprising a frequency decomposition module and a first classification neural network, a module for detecting voices within the audio signals by virtue of a second classification neural network, and a selective attenuation module supplying audio signals corresponding to the audio signals selectively attenuated in amplitude, no attenuation being applied in the presence of voice signals within the audio signals, and otherwise, an attenuation being applied in the presence of breathing noise within the audio signals. Thus, the intelligibility of the communications involving a pilot wearing the oxygen mask is improved.

### CROSS-REFERENCES TO RELATED APPLICATIONS

This application claims the benefit of the French patent application No. 1871614 filed on Nov. 20, 2018, the entire disclosures of which are incorporated herein by way of reference.

### FIELD OF THE INVENTION

The present invention relates to a method and a system for attenuating audio signals for an oxygen mask microphone designed to be used by aircraft pilots.

### BACKGROUND OF THE INVENTION

The cockpits of aircraft are equipped with oxygen masks in order to allow the pilots to breathe when a fire or a depressurization occurs. These oxygen masks are fitted with microphones in order to allow the pilots to communicate. However, when the aircraft pilots are wearing their oxygen mask, the quality of the communication may be degraded by the sound level of their breathing This problem arises mainly from the breathing noise of a pilot who is not speaking while someone else (e.g., another pilot, etc.) is talking.

It is desirable to overcome this drawback of the prior art. It is thus desirable to improve the quality of communication when the aircraft pilots are wearing their oxygen masks. It is furthermore desirable to modify as little as possible the voice signals captured by the microphones of the oxygen masks, while at the same time improving the intelligibility of the communications.

### SUMMARY OF THE INVENTION

One aim of the present invention is to provide a system for processing audio signals for an oxygen mask microphones of aircraft, the audio signal processing system being designed to receive audio signals x(t) which are captured by the oxygen mask microphone, characterized in that the audio signal processing system comprises: a breathing noise detection module within the audio signals x(t) comprising a frequency decomposition module carrying out a frequency decomposition of the audio signals x(t) and a first classification neural network configured for detecting the presence or otherwise of breathing noise within the audio signals x(t) based on the frequency decomposition of the audio signals x(t); a module for detecting voice signals within the audio signals x(t) comprising a second classification neural network configured for detecting a presence or otherwise of breathing noise within the audio signals x(t) based on the voice signals within the audio signals x(t); and a selective attenuation module of the audio signals x(t) supplying audio signals x_{f}(t) corresponding to the audio signals x (t) selectively attenuated in amplitude, no attenuation being applied in the presence of voice signals within the audio signals x(t), and otherwise, an attenuation being applied in the presence of breathing noise within the audio signals x(t). Thus, the intelligibility of the communications involving a pilot wearing the oxygen mask is improved.

According to one particular embodiment, in the absence of voice signals within the audio signals x(t), the selective attenuation module of the audio signals x(t) applies a first attenuation by a factor F1 to the audio signals x(t) in the presence of breathing noise within the audio signals x(t) and applies a second attenuation by a factor F2 to the audio signals x(t) in the absence of breathing noise within the audio signals x(t), the factor F2 being strictly less than the factor F1.

According to one particular embodiment, the audio signals x_{f}(t) are defined as follows:

$${x}_{f} ( t ) = \frac{x ( t ) \cdot ( {y}_{1} + 0.15 ) * ( {y}_{2} + 0.05 )}{1.2075}$$

where y₁ represents an output of the voice detection module and y₂ represents an output of the breathing noise detection module, and where y₁ takes the value ‘0’ in the absence of voice signals within the audio signals x(t) and ‘1’ otherwise, and y₂ takes the value ‘1’ in the absence of breathing noise within the audio signals x(t) and ‘0’ otherwise.

According to one particular embodiment, the frequency decomposition module applies a short-time Fourier transform to the audio signals x(t) and supplies to the first classification neural network a frequency decomposition magnitude matrix resulting from the application of the short-time Fourier transform.

According to one particular embodiment, the first classification neural network is a convolution neural network.

According to one particular embodiment, the second classification neural network is a neural network with long- and short-term memory.

According to one particular embodiment, the audio signal processing system is timed by cycles, the voice signal detection module furthermore comprises a post-processing module and, when the second classification neural network detects the presence of a voice in any cycle, the post-processing module is configured for indicating to the selective attenuation module the presence of voice signals during a predefined quantity N>1 of consecutive cycles.

According to one particular embodiment, each cycle has a duration of 62.5 milliseconds and N=5.

Another aim of the present invention is to provide an oxygen mask for aircraft comprising a microphone and an audio signal processing system such as the aforementioned system, in any one of its embodiments.

Another aim of the present invention is to provide an aircraft comprising at least one oxygen mask designed to be worn by at least one respective pilot of the aircraft, each oxygen mask comprising a microphone designed to capture the voice of the pilot wearing the oxygen mask, the aircraft furthermore comprising, for each oxygen mask, an audio signal processing system such as the aforementioned system, in any one of its embodiments.

Another aim of the present invention is to provide a method for processing audio signals for a microphone of an aircraft oxygen mask, the method comprising a step for receiving audio signals x(t) which are captured by the oxygen mask microphone, characterized in that the method furthermore comprises the following steps: detection of breathing noise within the audio signals x(t) by virtue of a frequency decomposition of the audio signals x(t) and detection of the presence or otherwise of breathing noise within the audio signals x(t) by a first classification neural network using the frequency decomposition of the audio signals x(t); detection of voice signals within the audio signals x(t) by a second classification neural network based on the voice signals within the audio signals x(t); and, selective attenuation of the audio signals x(t) so as to supply audio signals x_{f}(t) corresponding to the audio signals x(t) selectively attenuated in amplitude, no attenuation being applied in the presence of voice signals within the audio signals x(t), and otherwise, an attenuation being applied in the presence of breathing noise within the audio signals x(t).

Another aim of the present invention is to provide a computer program product, which may be stored on a medium and/or downloaded from a communications network, in order to be read by a processor of the system described hereinabove. This computer program comprises instructions for implementing the aforementioned method, when the program is executed by the processor. Another aim of the present invention is to provide an information storage medium on which such a computer program is stored.

### BRIEF DESCRIPTION OF THE DRAWINGS

The aforementioned features of the invention, together with others, will become more clearly apparent upon reading the following description of at least one exemplary embodiment, the description being presented in relation with the appended drawings, amongst which:

FIG. 1 shows a side view of an aircraft equipped with an audio signal processing system for an oxygen mask microphone;

FIG. 2 illustrates schematically a logical arrangement of the audio signal processing system according to one particular embodiment;

FIG. 3 illustrates schematically a hardware arrangement of the audio signal processing system according to one particular embodiment; and

FIG. 4 illustrates schematically a flow diagram of an algorithm for processing audio signals according to one particular embodiment.

### DETAILED DESCRIPTION OF THE PREFERRED EMBODIMENTS

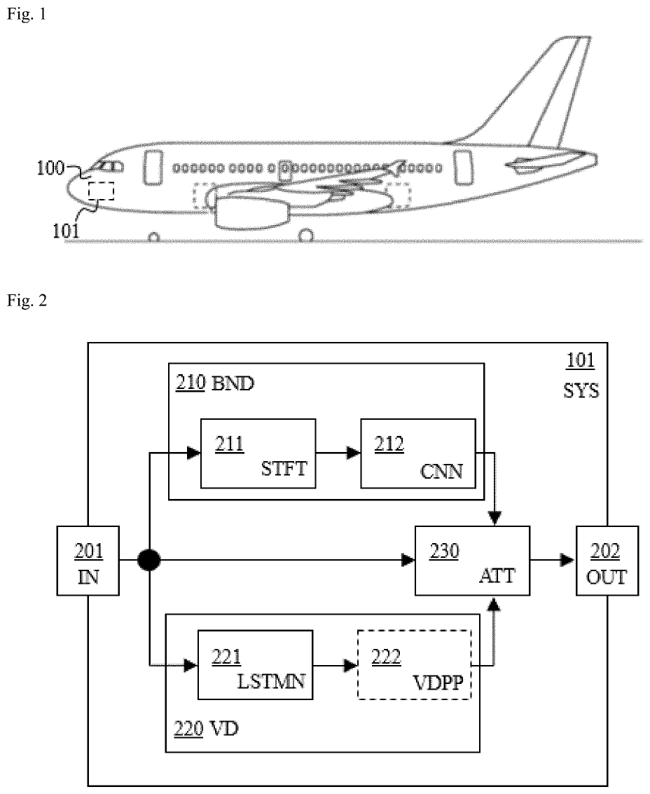

FIG. 1 illustrates schematically a side view of an aircraft 100. The aircraft 100 comprises a cockpit in which at least one pilot is designed to be installed in order to fly the aircraft 100. Amongst an assembly of equipment at his disposal, each pilot possesses an oxygen mask designed to be worn by the pilot when a predefined emergency situation occurs, such as, for example, a fire or a depressurization. Preferably, the aircraft 100 comprises a plurality of oxygen masks for a plurality of respective pilots. Each oxygen mask is equipped with a microphone allowing the voice of the pilot wearing the oxygen mask to be captured. Such oxygen masks are more particularly masks of the FFQDM (Full Face Quick Donning Mask) type.

The aircraft 100 furthermore comprises an audio signal processing system SYS 101 for each oxygen mask. The audio signal processing system SYS 101 is connected to the microphone of the oxygen mask with which the audio signal processing system SYS 101 is associated. The audio signal processing system SYS 101 applies a selective attenuation to the audio signal coming from the microphone, such as detailed in the following. The audio signal processing system SYS 101 is connected in series with the microphone of the oxygen mask with which the audio signal processing system SYS 101 is associated. The audio signal processing system SYS 101 is therefore transparent for the rest of the communications system in which the microphone of the oxygen mask, with which the audio signal processing system SYS 101 is associated, is generally used.

The audio signal processing system SYS 101 may be integrated into each oxygen mask with which it is associated. The audio signal processing system SYS 101 may, as a variant, be integrated into the equipment of the cockpit and the microphone of the oxygen mask is then connected via a dedicated cable to the audio signal processing system SYS 101 associated with the oxygen mask. This arrangement improves the clarity of communication between aircraft pilots, or between aircraft pilots and ground staff.

In another variant embodiment, the audio signal processing system SYS 101 may be remote with respect to the cockpit of the aircraft 100. For example, the audio signal processing system SYS 101 is situated on the ground and an air-ground communication propagates the audio signals captured by the microphone of the oxygen mask from the cockpit of the aircraft 100 to the audio signal processing system SYS 101 associated with the oxygen mask. This arrangement improves the clarity of communication between aircraft pilots and ground staff.

FIG. 2 illustrates schematically a logical arrangement of the audio signal processing system SYS 101 according to one particular embodiment. This logical arrangement may be implemented in the form of corresponding hardware modules, for example by virtue of one or more components of the FPGA (Field-Programmable Gate Array) or ASIC (Application-Specific Integrated Circuit) type. This logical arrangement may be implemented in the form of software modules executed by a processor.

The arrangement in FIG. 2 comprises an input interface IN 201 via which the audio signal processing system SYS 101 receives audio signals coming from the microphone of the oxygen mask associated with the audio signal processing system SYS 101.

The arrangement in FIG. 2 also comprises an output interface OUT 202 via which the audio signal processing system SYS 101 supplies audio signals which correspond to the audio signals received via the input interface IN 201 after potentially being attenuated.

The timing of the audio signal processing system SYS 101 is by cycles of predefined duration T. The audio signal processing system SYS 101 therefore carries out an analysis of audio signals at each cycle and applies, at each cycle, a decision on selective attenuation of the audio signals received via the input interface IN 201. For example, the duration T is 62.5 milliseconds.

The arrangement in FIG. 2 comprises a breathing noise detection module BND 210, together with, in parallel, a voice detection module VD 220.

The breathing noise detection module BND 210 is configured for analyzing the audio signals received via the input interface IN 201 in order to detect within them the presence of noise from the breathing of the pilot through the oxygen mask. The breathing noise detection module BND 210 is configured for supplying at the output, for each cycle, information indicating whether breathing noise has been detected in the audio signals received via the input interface IN 201. Preferably, the breathing noise detection module 210 is configured for supplying at the output, for each cycle, a bit of value ‘0’ when breathing noise is thus detected, and of value ‘1’ otherwise.

The voice detection module VD 220 is configured for analyzing the audio signals received via the input interface IN 201 in order to detect within them the presence of signals from the voice of the pilot. The voice detection module VD 220 is configured for supplying at the output, for each cycle, information indicating whether voice signals have been detected in the audio signals received via the input interface IN 201. Preferably, the voice detection module VD 220 is configured for supplying at the output, for each cycle, a bit of value ‘1’ when voice signals are detected, and of value ‘0’ otherwise.

The outputs of the breathing noise detection module BND 210 and of the voice detection module VD 220 are connected to the input of a selective attenuation module ATT 230, which also receives at its input the audio signals received via the input interface IN 201. Depending on the outputs of the breathing noise detection module BND 210 and of the voice detection module VD 220, the selective attenuation module ATT 230 is configured for deciding to apply or not to apply an amplitude attenuation to the audio signals received via the input interface IN 201 and, if so, to decide which value of attenuation to apply. Thus:

when the voice detection module VD 220 notifies that voice signals are detected, the selective attenuation module ATT 230 supplies, via the output interface OUT 202, the audio signals received via the input interface IN 201;

when the voice detection module VD 220 notifies that voice signals are not detected, and that furthermore the breathing noise detection module BND 210 notifies that breathing noise has been detected, the selective attenuation module ATT 230 supplies, via the output interface OUT 202, the audio signals received via the input interface IN 201 attenuated in amplitude by a factor F1; and

preferably, when the voice detection module VD 220 notifies that voice signals are not detected, and that furthermore the breathing noise detection module BND 210 notifies that breathing noise has not been detected, the selective attenuation module ATT 230 supplies, via the output interface OUT 202, the audio signals received via the input interface IN 201 attenuated in amplitude by a factor F2, the factor F2 being strictly less than the factor F1.

In other words, the selective attenuation module ATT 230 carries out, in the absence of voice signals, a higher attenuation when breathing noise is detected than when only background noise subsists, this background noise notably corresponding to the distribution of oxygen into the oxygen mask.

In one particular embodiment, the selective attenuation module ATT 230 supplies audio signals x_{f}(t) at the output defined as follows:

$${x}_{f} ( t ) = \frac{x ( t ) \cdot ( {y}_{1} + 0.15 ) * ( {y}_{2} + 0.05 )}{1.2075}$$

where x(t) represents the audio signals received via the input interface IN 201, y₁ represents the output of the voice detection module VD 220 and y₂ represents the output of the breathing noise detection module BND 210,

where y₁ takes the value ‘0’ in the absence of voice signals and ‘1’ in the presence of voice signals, and y₂ takes the value ‘1’ in the absence of breathing noise and ‘0’ in the presence of breathing noise, the values y₁ and y₂ being respectively re-evaluated by the voice detection module VD 220 and the breathing noise detection module BND 210 at each cycle.

Thus, the sound volume of the audio signals captured by the oxygen mask microphone is reduced by a factor approximately equal to 8 when there is neither the presence of voice signals nor the presence of breathing noise, i.e., a reduction of approximately 9 dB. The sound volume of the audio signals captured by the oxygen mask microphone is reduced by a factor equal to 160 when there is a presence of breathing noise, i.e., a reduction of 22 dB. When there is a presence of voice signals, the output audio signal is equal to the input audio signal. The audio signal processing system SYS 101 does not therefore modify the voice signals captured by the oxygen mask microphones, but significantly attenuates the background noise and breathing noise when the pilot in question is not speaking, which allows any other interlocutor involved in the communication to be better heard.

The breathing noise detection module BND 210 comprises a first classification neural network 212 and a frequency decomposition module 211 which carries out a frequency decomposition of the audio signals received via the input interface IN 201 as a function of time. In other words, the frequency decomposition module 211 obtains time-frequency distribution information TFD corresponding to the audio signals received via the input interface IN 201, subsequently used by the first classification neural network 212 for determining whether breathing noise is present in the audio signals received via the input interface IN 201.

In one particular embodiment, the frequency decomposition module 211 is configured for applying a short-time Fourier transform STFT, also known as local Fourier transform or else sliding-window Fourier transform, to the audio signals received via the input interface IN 201. The audio signals received via the input interface IN 201 are processed over a sliding window of duration Tsw. Various successive time slots of the sliding window are used over the duration T of each cycle, with an overlap of duration To of one time slot of the sliding window with respect to the preceding time slot. The short-time Fourier transform thus allows two frequency decomposition matrices to be obtained at the output as a function of time (one column for each time slot of the sliding window during the cycle in question): a first matrix supplying information on magnitude and a second matrix supplying information on phase. Only the first matrix supplying information on magnitude is used by the first classification neural network 212 for determining whether breathing noise is present in the audio signals received via the input interface IN 201. One advantage of the short-time Fourier transform STFT is a good ratio efficiency—cost of implementation and of execution.

In one variant embodiment, the frequency decomposition module 211 is configured for applying a Hilbert transform to the audio signal received via the input interface IN 201. The Hilbert transform allows a new form of time-domain signal to be obtained, on which the frequency decomposition module 211 calculates intrinsic-mode functions IMF starting from the highest frequency of the frequency spectrum in question down to the lowest frequency of the spectrum in question. As a reminder, an IMF function complies with the following requirements: within the interval of time in question, the number of extrema and the number of zero-crossings must be equal or differ at the most by one unit; and, at any point, the average value of the envelope defined by the local maxima and that defined by the local minima is equal to zero. As soon as a function IMF is obtained, it is subtracted from the signal to be processed and a new function IMF is sought on the remainder after subtraction. The amplitudes of each function IMF thus obtained allow two frequency decomposition matrices to be filled in as a function of time: a first matrix supplying information on magnitude for each function IMF and a second matrix supplying information on phase for each function IMF. As in the case of the short-time Fourier transform STFT, only the first matrix supplying information on magnitude is used by the first classification neural network 212 for determining whether breathing noise is present in the audio signals received via the input interface IN 201. Compared with the short-time Fourier transform STFT, the Hilbert transform offers a higher precision of decomposition, at a higher cost in terms of implementation and of execution.

In another variant embodiment, the frequency decomposition module 211 is configured for applying a wavelet transform. It is recalled that a wavelet Ψ is a time-domain function, which therefore depends on the time t, and which meets the following requirements:

$$\{ \begin{matrix}{\int }_{- ∞}^{+ ∞} Ψ ( t ) dt = 0 \\ {‖ Ψ ( t ) ‖}^{2} = {\int }_{- ∞}^{+ ∞} Ψ ( t ) {Ψ}^{*} ( t ) dt\end{matrix}$$

A wavelet is based on two parameters: a time-domain parameter u, called translation parameter, and a scale parameter s which describes a modification in frequency, which may be expressed as follows:

$${Ψ}_{s , u} ( t ) = \frac{1}{\sqrt{s}} Ψ ( \frac{t - u}{s} )$$

By accordingly applying a continuous wavelet transform to the audio signals x(t) received via the input interface IN 201, a complex matrix X of s rows and u columns may be formed in the following manner

$$X ( s , u ) = {\int }_{- ∞}^{+ ∞} x ( t ) * {Ψ}_{s , u}^{*} ( t ) dt = \frac{1}{\sqrt{s}} {\int }_{- ∞}^{+ ∞} x ( t ) * {Ψ}^{*} ( \frac{t - u}{s} ) dt$$

Information on magnitude ⊕X(s, u)| is then obtained for each cell X(s, u) of the complex matrix X. Information on phase angle (X(s, u)) may also be obtained for each cell X(s, u) of the complex matrix X. However, as in the case of the short-time Fourier transform STFT, only the information on magnitude is used by the first classification neural network 212 for determining whether breathing noise is present in the audio signals received via the input interface IN 201. Compared with the alternatives previously described, the wavelet transform offers an even greater precision, to the detriment however of the cost of implementation and of execution notably due to the redundancies in the matrix components.

In one particular embodiment, the first classification neural network 212 is a convolutional neural network CNN. As a reminder, the convolutional neural network CNN is a type of network of acyclic artificial neurons, in which the pattern of connections between the neurons is inspired by the visual cortex of animals. This type of neural network is particularly well adapted to the recognition of patterns, notably in imaging technology. It allows an easy and efficient recognition of breathing noise, more particularly when it is coupled to the short-time Fourier transform STFT.

The voice detection module VD 220 comprises a second classification neural network 221 and potentially a post-processing VDPP (Voice Detection Post-Processing) module 222.

In one particular embodiment, the second classification neural network 221 is a network with long short-term memory LSTM. As a reminder, the network with long short-term memory LSTM is a recurrent neural network RNN whose input is partially dependent on inputs and/or on outputs from preceding iterations. By construction, the network with long short-term memory LSTM handles short-term information and also long-term information. It is then particularly well adapted to the processing of voice signals, owing to the short-term nature of their spectral characteristics and to the long-term nature of their frequency modulations.

In one variant embodiment, the second classification neural network 221 is a convolutional neural network CNN. It should be noted that the first classification neural network 212 may also be a network with long short-term memory LSTM.

Neural networks require a learning phase using learning databases. Each learning database comprises a multitude of input data sets for which the data expected at the output of the neural network in question is known and listed in the learning database. For each set of input data supplied to the neural network in question, the data at the output of the neural network is compared with the output data expected in theory, and the error observed is propagated back via the neural network. At each layer of the neural network, the link weights between the neurons are updated by the back-propagation. Various types of learning algorithms may be used, depending on the activation function of the neurons and on the type of data being processed. The learning phase is completed by a validation phase so as to refine the internal structure of the neural network (this time without acting on the link weights).

A learning database for the first classification neural network 212 is composed of samples of breathing audio signals through various oxygen masks used in the aircraft on the market. The audio signals of the learning database have a sampling frequency appropriate to the audio signals subsequently processed by the first classification neural network 212, for example 8 kHz. These audio signals of the learning database may be noisy or otherwise.

Another learning database for the second classification neural network 212 is composed of samples of audio signals of various types of voice. This learning database may be populated by virtue of recordings made by the ATC (Air Traffic Control) in its communications with aircraft pilots. These audio signals of the learning database may come from reference databases containing audio recordings of female and male voices speaking with diverse accents, such as for example the CMU (Carnegie Mellon University) Artic databases of the Festvox project. Such databases often have the advantage of being free from background noise, which improves the efficiency of learning. The audio signals of this learning database also have a sampling frequency appropriate to the audio signals subsequently processed by the second classification neural network 221, for example 8 kHz.

A validation database is used to carry out the validation phase. For example, recordings coming from one or more CVR (Cockpit Voice Recorder) units, also referred to as the “black box” of aircraft, may be used since recordings are stored there of everything that is said in the cockpit via the various microphones available to the pilots, hence including the microphones in their oxygen masks. Here again, the sampling frequency used is appropriate to the audio signals subsequently processed by the first 212 and second 221 classification neural networks, for example 8 kHz. Other signals from the recordings made by the air traffic control ATC and/or other signals coming from the reference databases may also be used for the validation phase.

The audio signals stored in the aforementioned databases are dimensioned according to the predefined duration T of the timing cycles of the audio signal processing system SYS 101. For a sampling frequency of 8 kHz and a duration T of 62 5 milliseconds, this constitutes audio signals composed of 500 values, each being marked in the corresponding database with information indicating whether breathing noise is present or not and with information indicating whether voice signals are present or not.

The post-processing module VDPP 222 allows the output value of the second classification neural network 221 to be modified under certain conditions. More particularly, when the second classification neural network 221 detects the presence of a voice in any cycle, the output of the post-processing module VDPP 222 indicates the presence of voice signals to the selective attenuation module ATT 230 during a predefined quantity N>1 of consecutive cycles. In one particular embodiment, N=5 for a cycle duration T equal to 62.5 milliseconds, which is equivalent to 312.5 milliseconds. Such a duration avoids the decrease in the sound volume between the words being pronounced. The result heard is then smoother and more natural, because there will be fewer modifications of the amplitude of the signal at the output of the audio signal processing system SYS 101.

It has implicitly been considered hereinabove that, in the presence of voice signals, the breathing noises captured by the same oxygen mask microphone are negligible and not detected. If, however this were not the case, the selective attenuation module ATT 230 only takes into account the output of the breathing noise detection module 210 when the voice detection module VD 220 does not indicate the presence of voice signals, and considers that there is an absence of breathing noise otherwise.

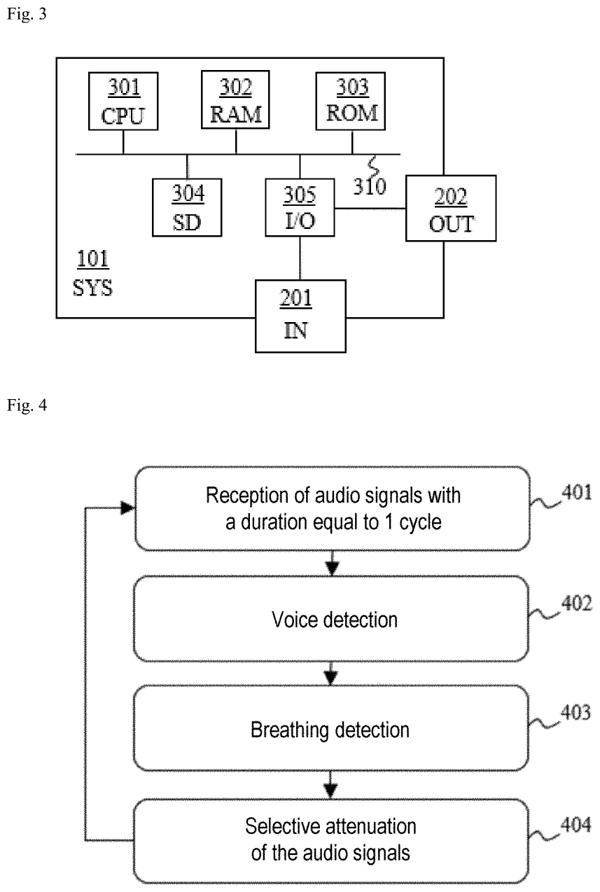

FIG. 3 illustrates schematically one arrangement of the audio signal processing system SYS 101. FIG. 3 thus schematically illustrates the input interface IN 201 and output interface OUT 202. In addition, in FIG. 3, the audio signal processing system SYS 101 comprises, connected via a communications bus 310: a processor 301; a volatile memory 302; a non-volatile memory 303, for example of the ROM (Read Only Memory) or EEPROM (Electrically-Erasable Programmable Read Only Memory) type; a storage unit 304, such as a hard disk HDD (Hard Disk Drive), or a storage medium reader, such as an SD (Secure Digital) card reader; an input-output interface controller 305 controlling the input IN 201 and output OUT 202 interfaces.

The processor 301 is capable of executing instructions loaded into the volatile memory 302 from the non-volatile memory 303, from an external memory, from a storage medium (such as an SD card), or from a communications network. When the audio signal processing system SYS 101 is powered up, the processor 301 is capable of reading instructions from the volatile memory 302 and of executing them. These instructions form a computer program causing the implementation, by the processor 301, of all or part of the algorithm and of the steps described hereinafter in relation with FIG. 4.

All or part of the algorithm and of the steps described hereinafter in relation with FIG. 4, as well as all or part of the logical arrangement in FIG. 2, may thus be implemented in software form by execution of a set of instructions by a programmable machine, for example a processor of the DSP (Digital Signal Processor) or a microcontroller, or may be implemented in hardware form by a machine or a dedicated component, for example an FPGA or ASIC component. Generally speaking, the audio signal processing system SYS 101 comprises electronic circuitry adapted and configured for implementing, in software and/or hardware form, the algorithm and the steps described hereinafter in relation with FIG. 4.

FIG. 4 illustrates schematically a flow diagram of an algorithm for processing audio signals according to one embodiment of the present invention.

In a step 401, the audio signal processing system SYS 101 receives audio signals x(t), recorded by the microphone of the oxygen mask with which the audio signal processing system SYS 101 is associated, with a duration T of one cycle.

In a step 402, the audio signal processing system SYS 101 carries out, by virtue of the second classification neural network 221, a detection of voice signals within the audio signals x(t) received. The audio signal processing system SYS 101 then identifies whether voice signals are present or not in the audio signals x(t) received. This aspect has already been detailed in relation with FIG. 2.

In a step 403, the audio signal processing system SYS 101 carries out, by virtue of the first classification neural network 212, after determination of the information on time-frequency distribution TFD of the audio signals x(t) received, a detection of breathing noise within the audio signals x(t) received. The audio signal processing system SYS 101 then identifies whether breathing noise is present or not in the audio signals x(t) received. This aspect has also already been detailed in relation with FIG. 2.

In a step 404, the audio signal processing system SYS 101 carries out a selective attenuation of the audio signals x(t) in order to supply audio signals x_{f}(t) corresponding to the audio signals x (t) potentially attenuated in amplitude, depending on the presence or otherwise of breathing noise within the audio signals x(t) and on the presence or otherwise of voice signals within the audio signals x(t). This aspect has also already been detailed in relation with the FIG. 2. Subsequently, the step 401 is repeated for a new cycle.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A system for processing audio signals for a microphone of an aircraft oxygen mask, the audio signal processing system configured to receive audio signals which are captured by the oxygen mask microphone, wherein the audio signal processing system comprises:
a module for detecting breathing noise within the audio signals comprising a frequency decomposition module carrying out a frequency decomposition of the audio signals and a first classification neural network configured to detect a presence or otherwise of breathing noise within the audio signals based on the frequency decomposition of the audio signals;
a module for detecting voice signals within the audio signals comprising a second classification neural network configured to detect a presence or otherwise of the voice signals from the audio signals; and
a module for selective attenuation of the audio signals supplying audio signals corresponding to the audio signals selectively attenuated in amplitude, no attenuation being applied in the presence of voice signals within the audio signals, and otherwise, an attenuation being applied in the presence of breathing noise within the audio signals.

2. The audio signal processing system according to claim 1, wherein, in an absence of voice signals within the audio signals, the module for selective attenuation of the audio signals applies a first attenuation by a factor F1 to the audio signals in the presence of breathing noise within the audio signals and applies a second attenuation by a factor F2 to the audio signals in an absence of breathing noise within the audio signals, the factor F2 being strictly less than the factor F1.

3. The audio signal processing system according to claim 1, wherein the audio signals are defined as follows:
$${x}_{f} ( t ) = \frac{x ( t ) * ( {y}_{1} + 0.15 ) * ( {y}_{2} + 0.05 )}{1.2075}$$
where y₁ represents an output of the voice detection module and y₂ represents an output of the breathing noise detection module, and
where y₁ takes a value ‘0’ in an absence of voice signals within the audio signals and ‘1’ otherwise, and y₂ takes a value ‘1’ in an absence of breathing noise within the audio signals and ‘0’ otherwise.

4. The audio signal processing system according to any claim 1, wherein the frequency decomposition module applies a short-time Fourier transform to the audio signals and supplies to the first classification neural network a frequency decomposition magnitude matrix resulting from an application of the short-time Fourier transform.

5. The audio signal processing system according to claim 1, wherein the first classification neural network is a convolutional neural network.

6. The audio signal processing system according to claim 1, wherein the second classification neural network is a neural network with long short-term memory.

7. The audio signal processing system according to claim 1, wherein a timing of the audio signal processing system is by cycles, the voice detection module furthermore comprises a post-processing module and, when the second classification neural network detects the presence of a voice in any cycle, the post-processing module is configured to indicate to the selective attenuation module the presence of voice signals during a predefined quantity N>1 of consecutive cycles.

8. The audio signal processing system according to claim 7, wherein each cycle has a duration of 62.5 milliseconds and N=5.

9. An oxygen mask for aircraft comprising a microphone and an audio signal processing system according to claim 1.

10. An aircraft comprising:
at least one oxygen mask configured to be worn by at least one respective pilot of the aircraft, each oxygen mask comprising a microphone designed to capture a voice of the pilot wearing the oxygen mask,
for each oxygen mask, an audio signal processing system according to claim 1.

11. A method for processing audio signals for a microphone of an aircraft oxygen mask, the method comprising:
receiving audio signals which are captured by the oxygen mask microphone,
detecting breathing noise within the audio signals by virtue of a frequency decomposition of the audio signals and detection of a presence or otherwise of breathing noise within the audio signals by a first classification neural network based on a frequency decomposition of the audio signals;
detecting voice signals within the audio signals by a second classification neural network from the audio signals; and
selectively attenuating the audio signals to supply audio signals corresponding to the audio signals selectively attenuated in amplitude, no attenuation being applied in a presence of voice signals within the audio signals, and otherwise, an attenuation being applied in the presence of breathing noise within the audio signals.

12. A computer program product, comprising instructions driving an execution, by a processor, of the method according to claim 11, when the instructions are executed by the processor.

13. A storage medium, storing a computer program comprising instructions driving an execution, by a processor, of the method according to claim 11, when the instructions are read and executed by the processor.
